(19) United States
(12) Patent Application Publication
(10) Pub. No.: US 2024/0185972 A1
(43) Pub. Date: Jun. 6, 2024

(54) Line of Therapy Identification from Clinical Documents

(71) Applicant: Bristol-Myers Squibb Company, Princeton, NJ (US)

(72) Inventors: Sazedul Alam, Lawrenceville, NJ (US); Gengyuan Liu, Lawrenceville, NJ (US); Devansh Agarwal, Lawrenceville, NJ (US); Md Shamsuzzaman, Lawrenceville, NJ (US)

(73) Assignee: Bristol-Myers Squibb Company, Princeton, NJ (US)

(21) Appl. No.: 18/526,197

(22) Filed: Dec. 1, 2023

Related U.S. Application Data
(60) Provisional application No. 63/429,485, filed on Dec. 1, 2022.

(51) Int. Cl. G16H 15/00; G16H 10/20; G16H 50/70

(52) U.S. Cl. CPC G16H 15/00; G16H 10/20; G16H 50/70

(57) ABSTRACT

A method includes receiving input data including unstructured text representing one or more sequences of terms. For each respective sequence of terms, the method includes generating a corresponding line of therapy (LoT) pseudo-label indicating whether the respective sequence of terms includes LoT information, generating a corresponding LoT indicator predicting whether the respective sequence of terms includes LoT information, and determining a corresponding LoT indication loss based on the corresponding LoT pseudo-label and the corresponding LoT indicator. The method also includes fine-tuning a pre-trained transformer model based on the LoT indication losses determined for the one or more sequences of terms.

### CROSS REFERENCE TO RELATED APPLICATIONS

This U.S. Patent Application claims priority under 35 U.S.C. § 119(e) to U.S. Provisional Application 63/429,485, filed on Dec. 1, 2022. The disclosure of this prior application is considered part of the disclosure of this application and is hereby incorporated by reference in its entirety.

### TECHNICAL FIELD

This disclosure relates to line of therapy identification from clinical documents.

### BACKGROUND

Clinical trial design (CTD) is a significant part of drug development as it impacts clinical trial length, protocol, patient enrollment, clinical endpoints, and comparator agents required. Knowledge from earlier clinical trials for a same drug, or a different drug, can help in the CTD process. A line of therapy (LoT) refers to the order of different treatments given to a patient during their disease progression. LoT is an important concept in the context of clinical trials as it may be relevant to the patient population for which a regulatory agency approves the drug. For example, regulatory agencies may grant approval for a first drug only to patients who have already failed the standard of care (SoC) therapy (e.g., first line of therapy), in which case the approval of the first drug would be referred to as second line of therapy approval. Similarly, if a second drug is only approved for patients for patients who have failed the SoC therapy and also therapy with the first drug, approval of the second drug would be referred to as a third line of therapy approval.

### SUMMARY

One aspect of the disclosure provides a computer-implemented method that when executed on data processing hardware causes the data processing hardware to perform operations for using weak supervision deep learning models for line of therapy identification from clinical documents. The operations include receiving input data including unstructured text representing one or more sequences of terms. For each respective sequence of terms, the operations include: generating, using regular expression rules, a corresponding line of therapy (LoT) pseudo-label indicating whether the respective sequence of terms includes LoT information, generating, using a pre-trained transformer model, a corresponding LoT indicator predicting whether the respective sequence of terms includes LoT information, and determining a corresponding LoT indication loss based on the corresponding LoT pseudo-label and the corresponding LoT indicator. The operations also include fine-tuning the pre-trained transformer model based on the LoT indication losses determined for the one or more sequences of terms. Implementations of the disclosure may include one or more of the following optional features. In some implementations, the operations further include for each respective sequence of terms: generating, using the regular expression rules, a corresponding classification pseudo-label indicating a classification of the respective sequence of terms, generating, using the pre-trained transformer model, a corresponding LoT classification predicting the classification of the respective sequence of terms, and determining a corresponding LoT classification loss based on the corresponding classification pseudo-label and the corresponding LoT classification, and fine-tuning the pre-trained transformer model based on the LoT classification losses determined for the one or more sequences of terms. In these implementations, the classification of the respective sequence of terms indicates a particular LoT step from a series of LoT steps associated with the respective sequence of terms. The input data may include a plurality of clinical trial documents that includes the unstructured text.

In some examples, the operations further include updating the regular expression rules based on the corresponding LoT classification loss. The pre-trained transformer model includes a Bidirectional Encoder from Transformers for Biomedical Text Mining (BioBERT) model pre-trained on a corpus of biomedical text data. Here, the pre-trained BioBERT model may include a stack of multi-headed self-attention layers. In some implementations, the operations further include fine-tuning the pre-trained transformer model using an aggregation of training data including human annotated labels, labels generated using the regular expression rules, and labels generated by the pre-trained transformer model. In some examples, the operations further include storing the fine-tuned transformer model in memory hardware in communication with the data processing hardware. The operations may further include transmitting, via a network, the fine-tuned transformer model to one or more computing devices.

Another aspect of the disclosure provides a system that includes data processing hardware and memory hardware storing instructions that when executed on the data processing hardware causes the data processing hardware to perform operations. The operations include receiving input data including unstructured text representing one or more sequences of terms. For each respective sequence of terms, the operations include: generating, using regular expression rules, a corresponding line of therapy (LoT) pseudo-label indicating whether the respective sequence of terms includes LoT information, generating, using a pre-trained transformer model, a corresponding LoT indicator predicting whether the respective sequence of terms includes LoT information, and determining a corresponding LoT indication loss based on the corresponding LoT pseudo-label and the corresponding LoT indicator. The operations also include fine-tuning the pre-trained transformer model based on the LoT indication losses determined for the one or more sequences of terms.

Implementations of the disclosure may include one or more of the following optional features. In some implementations, the operations further include for each respective sequence of terms: generating, using the regular expression rules, a corresponding classification pseudo-label indicating a classification of the respective sequence of terms, generating, using the pre-trained transformer model, a corresponding LoT classification predicting the classification of the respective sequence of terms, and determining a corresponding LoT classification loss based on the corresponding classification pseudo-label and the corresponding LoT classification, and fine-tuning the pre-trained transformer model based on the LoT classification losses determined for the one or more sequences of terms. In these implementations, the classification of the respective sequence of terms indicates a particular LoT step from a series of LoT steps associated with the respective sequence of terms. The input data may include a plurality of clinical trial documents that includes the unstructured text.

In some examples, the operations further include updating the regular expression rules based on the corresponding LoT classification loss. The pre-trained transformer model includes a Bidirectional Encoder from Transformers for Biomedical Text Mining (BioBERT) model pre-trained on a corpus of biomedical text data. Here, the pre-trained BioBERT model may include a stack of multi-headed self-attention layers. In some implementations, the operations further include fine-tuning the pre-trained transformer model using an aggregation of training data including human annotated labels, labels generated using the regular expression rules, and labels generated by the pre-trained transformer model. In some examples, the operations further include storing the fine-tuned transformer model in memory hardware in communication with the data processing hardware. The operations may further include transmitting, via a network, the fine-tuned transformer model to one or more computing devices.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system executes machine learning components to process input data.

FIGS. 2A and 2B are schematic views of a pre-training stage and fine-tuning stage for training a BioBERT model.

FIG. 3 is a schematic view of an example BioBERT model.

FIG. 4 is a process flow diagram of an example training process for training the one or more machine learning components.

FIG. 5 is a table providing descriptions of different trained multi-label LoT classifiers.

FIG. 6 is a table providing the number of data samples for training and testing the different trained multi-label LoT classifiers from FIG. 5.

FIG. 7 is a table providing the distribution of LoT classes for training the different trained multi-label LoT classifiers from FIG. 5.

FIG. 8 is a table providing the number of data samples in each of the LoT classifications in the ground truth data.

FIG. 9 is a table showing the precision, recall, and F1 scores of the different trained multi-label LoT classifiers from FIG. 5.

FIG. 10 is a table that shows a coverage analysis for the different trained multi-label LoT classifiers from FIG. 5 using an unseen dataset.

FIG. 11 is a graph illustrating receiver operating characteristic curves showing the true positive rate versus the false positive rate for each of the LoT classification.

FIG. 12 is a schematic view of one or more other users executing the trained machine learning components.

FIG. 13 is a flowchart of an example arrangement of operations for a computer-implemented of using a weak supervision based deep learning model for LoT identification from clinical documents.

FIG. 14 is a schematic view of an example computing device that may be used to implement the systems and methods described herein.

Like reference symbols in the various drawings indicate like elements.

### DETAILED DESCRIPTION

The drug development process is a complex procedure where clinical trials are one of the lengthy and expensive components. Clinical trial documents include vast amounts of clinical information for different entities including disease entity, drug name, line of therapy, etc. Extracting and classifying these entities correctly may facilitate the design process of clinical trials. A line of therapy (LoT) is a series of ordered treatments given to patients during their disease progression. For example, a LoT for cancer treatment may include surgery first followed by chemotherapy and radiation. In this example, surgery is the first line of therapy, and chemotherapy and radiation are the second line of therapy. The LoT is an important concept used in many different clinical scenarios. For instance, in clinical trials, the LoT information of prior treatments can be used to include, or exclude, patients and then the current treatment may be placed as the next LoT. The LoT may also be reported in application materials for a regulatory approval of a drug. Moreover, doctors may choose a LoT for a patient based on his or her condition and provide treatment based on established guidelines pertinent to that LoT.

The LoT information may be used in different contexts such that the LoT information may be applied in several different applications. For instance, when designing a clinical trial of a drug, clinical trial designers may need to collect information from other clinical trials of similar drugs. In particular, the clinical trial designers may need to know which pivotal studies were used for the approval of a specific drug to leverage information from those pivotal studies. However, the regulatory approval documents may not explicitly mention the names and/or the National Clinical Trial (NCT) number of the corresponding clinical trial. This is known as the “trial matching problem,” where the clinical trial designer needs to find the clinical trials that were used for the approval of a specific drug. For instance, if a clinical trial designer wants to know which clinical trials were used for the regulatory approval of the drug “Accrufer” for the approval date of Jul. 25, 2019, then the answer may include the clinical trials “NCT01340872”, “NCT01352221”, and “NCT02968368”. To that end, different entities from the clinical trial documents may be used as well as the relationships between those entities that are common in the regulatory approval to identify the particular clinical trials. In some examples, the documents from the particular clinical trials used to approve a specific drug include entities such as disease entities, LoT, Treatment Regimen, name of drugs, co-treatments, a combination of drugs, bio-marker, disease sub-types, pathology, risk category, patient demographics, age cutoff, etc. Thus, correctly extracting and classifying these entities correctly may solve the trial matching problem. Moreover, different indications can be used for different approvals when a drug has multiple approvals. As such, the “unique indication identification problem” is when a clinical trial designer needs to know which indications were used for a particular approval. LoT information and other previously mentioned entities may similarly be extracted and classified to differentiate between multiple approvals of the same drug.

Despite the above, clinical trial documents include text in an unstructured text format. Thus, any LoT information included in the clinical trial documents is not structured. Consequently, regular expression and predefined rules may not always correctly identify the LoT information from the unstructured text format. Supervised deep learning models may be a useful alternative to extract the LoT information. However, developing a supervised deep learning model is cumbersome as a large amount of labeled data is required. Additionally, manually labeling LoT information correctly is costly and time-consuming and requires expert knowledge.

To that end, implementations herein are directed towards methods and systems for a weak supervision-based machine learning model for LoT identification from clinical documents. The method includes receiving input data that includes unstructured text representing one or more sequences of terms. For each respective sequence of terms, the method includes generating, using regular expression rules, a corresponding line of therapy (LoT) pseudo-label indicating whether the respective sequence of terms includes LoT information, generating, using a pre-trained transformer model, a corresponding LoT indicator predicting whether the respective sequence of terms includes LoT information, and determining a corresponding LoT indication loss based on the corresponding LoT pseudo-label and the corresponding LoT indicator. Thereafter, the method includes fine-tuning the pre-trained transformer model based on the LoT indication losses determined for the one or more sequences of terms.

Moreover, for each respective sequence of terms, the method may include generating, using the regular expression rules, a corresponding classification pseudo-label indicating a classification of the respective sequence of terms, generating, using the pre-trained transformer model, a corresponding LoT classification predicting the classification of the respective sequence of terms, and determining a corresponding LoT classification loss based on the corresponding classification pseudo-label and the corresponding LoT classification. Thereafter, the method includes fine-tuning the pre-trained transformer model based on the LoT classification losses determined for the one or more sequences of terms. As will become apparent, in some implementations, the pre-trained transformer model is fine-tuned on aggregation training data.

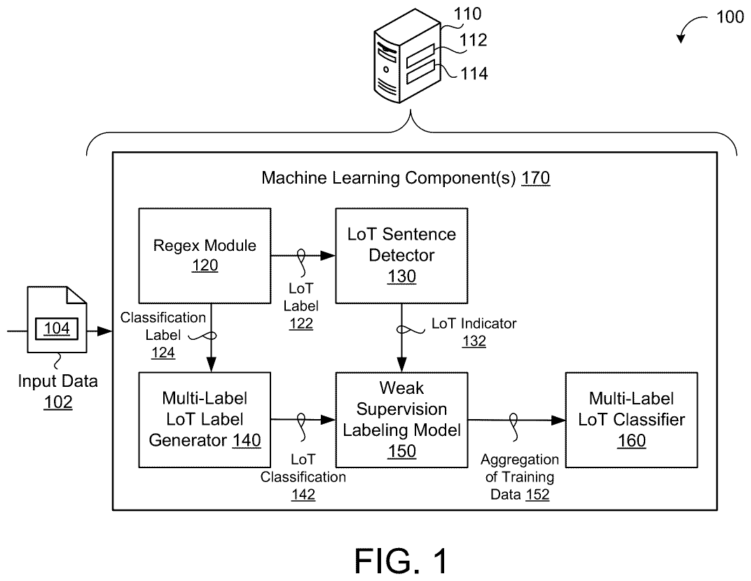

Referring now to FIG. 1, in some implementations, an example system 100 includes a processing system 110 which may be a single computer, multiple computers, a user device, or a distributed system (e.g., a cloud computing environment). The processing system 110 has fixed or scalable computing resources (e.g., data processing hardware) 112 and/or storage resources (e.g., memory hardware) 114. The data processing hardware 112 and the memory hardware 114 may reside on the user device and/or the cloud computing environment. The processing system 110 includes machine learning components 170 that process input data 102 to identify and extract a plurality of features to identify lines of therapy (LoT). The input data 102 may be stored at the memory hardware 114 of the processing system 110 or received, via a network or other communication channel, from another entity (e.g., another processing system).

The input data 102 may include training data such as a large dataset of clinical trial documents having unstructured text. That is, the input data 102 may include sequences of terms 104 (e.g., sentences) each of which may, or may not, include LoT information. The processing system 110 trains one or more of the machine learning components 170 by processing the input data 102. For example, the processing system 110 may train a multi-label LoT classifier 160 to identify and extract the sequence of terms 104 from the unstructured text of the input data 102 and classify different LoT for each extracted sequence of terms 104. Here, each sequence of terms 104 may correspond to a sentence from the input data 102 and represent LoT information. LoT information may be any textual information related to a one or more therapies received by a patient for a particular disease or treatment.

In some implementations, the machine learning components 170 include a regular expressions (regex) module 120, a LoT sentence detector 130, a multi-label LoT label generator 140, a weak supervision labeling model 150, and the multi-label LoT classifier 160. The regex module 120 is configured to annotate the sequences of terms 104 from the unstructured text of the input data 102. That is, not all of the sequences of terms 104 from unstructured text include LoT information, and thus, the regex module 120 determines whether each sequence of terms 104 includes LoT information or not. Moreover, the regex module 120 classifies sequences of terms 104 determined to include LoT information into a particular LoT class.

In some examples, the regex module 120 is a rule-based, deterministic model that generates pseudo-labels 122, 124 for each sequence of terms 104 from the input data 102. The sequence of terms 104 may include one or more words or sentences from the unstructured text of the input data 102. That is, the regex module 120 is a regex-based annotation module that generates the pseudo-labels 122, 124 by processing each sequence of terms 104 using an initial set of regular expression rules. A regular expression is a sequence of characters/terms that specifies a particular pattern in text. Thus, the regex module 120 generates the pseudo-labels 122, 124 determines whether the unstructured text of the input data 102 satisfies a matching threshold of any of the regular expression rules.

For example, when the regex module 120 determines that a respective sequence of terms 104 satisfies a matching threshold of at least one of the initial regular expression rules, the regex module 120 generates a corresponding LoT pseudo-label 122 indicating that the respective sequence of terms 104 includes LoT information. On the other hand, when the regex module 120 determines that a respective sequence of terms 104 fails to satisfy a matching threshold of at least one of the initial regular expression rules, the regex module 120 generates a corresponding LoT pseudo-label 122 indicating that the respective sequence of terms 104 does not include LoT information. Simply put, the LoT pseudo-label 122 indicates whether a particular sentence from the input data 102 includes LoT information or not.

For each sequence of terms 104 for which the regex module 120 generated a corresponding LoT pseudo-label 122 indicating that the sequence of terms 104 includes LoT information, the regex module 120 also generates a corresponding classification pseudo-label 124 indicating a classification of the respective sequence of terms 104. Here, the classification of the respective sequence of terms 104 terms indicates a particular LoT step from series of LoT steps associated with the respective sequence of terms 104. For instance, the respective sequence of terms 104 may be associated with a third LoT treatment from a series of five LoT treatments, and thus, the classification would indicate that the respective sequence of terms 104 is associated with the third LoT step. Thus, the regex module 120 may generate a corresponding classification pseudo-label 124 for a respective sequence of terms 104 if the respective sequence of terms 104 matches with a regular expression rule. For example, the regex module 120 may process the sequence of terms 104 corresponding to “For the treatment of adult patients with multiple myeloma as monotherapy, in patients who have received at least three prior lines of therapy including a proteasome inhibitor (PI) and an immunomodulatory agent or who are double-refractory to a PI and an immunomodulatory agent” and detect a keyword of ‘at least three’ and generate the corresponding LoT classification pseudo-labels 124 representing ‘LoT 4+’ as the classification for the sequence of terms 104. In this example, the ‘LoT 4+’ denotes that the LoT information from the sequence of terms 104 is associated with a fourth LoT step from a series of LoT steps.

The processing system 110 trains the LoT sentence detector 130 to identify whether the sequences of terms 104 from the input data 102 include LoT information or not. For instance, the LoT sentence detector 130 may include a pre-trained Bidirectional Encoder Representations from Transformers for Biomedical Text Mining (BioBert) model (e.g., pre-trained transformer model) 300 whereby the processing system 110 trains the BioBert model 300 to detect the presence of LoT information. Notably, the LoT sentence detector 130 is a neural network model that detects the presence of LoT information non-deterministically (in contrast to the regex module 120). The corresponding LoT pseudo-labels 122 generated for each respective sequence of terms 104 serve as corresponding ground-truth labels for training the LoT sentence detector 130.

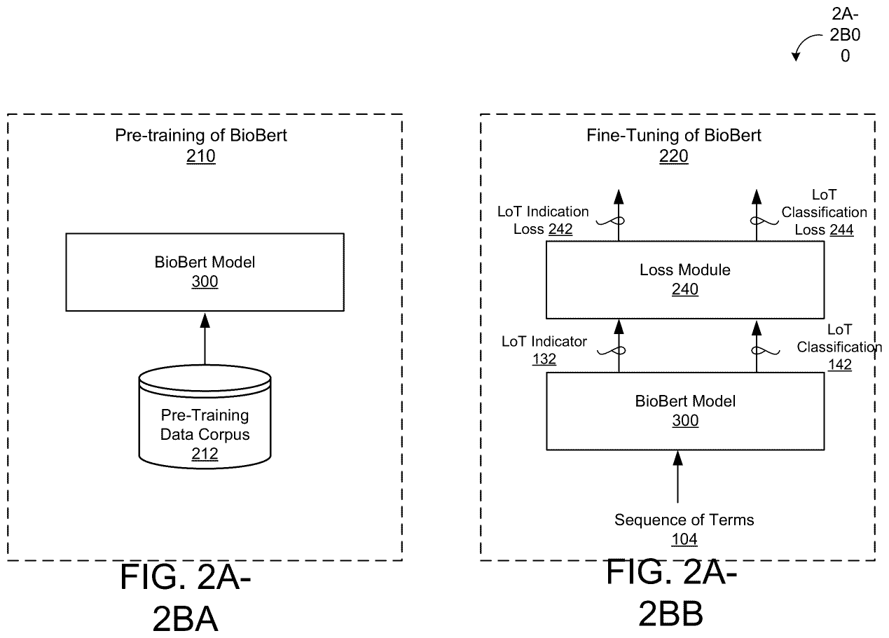

Referring now to FIGS. 2A and 2B, in some implementations, the processing system 110 executes a training process 200 to train the BioBert model 300. The BioBERT model 300 is a domain-specific language representation model. The training process 200 includes a pre-training stage 210 and a fine-tuning stage 220. In the pre-training stage 210, the training process 200 trains the BioBERT model 300 using a pre-training data corpus 212 that includes text from general websites and/or books. The pre-training stage 210 provides weight initialization for parameters of the BioBERT model 300. The BioBERT model 300 is also pre-trained using large-scale biomedical texts such as biomedical and life license literature abstracts and/or full-text articles in addition to, or in lieu of, the general website and/or books.

With continued reference to FIGS. 2A and 2B, after the BioBert model 300 is pre-trained during the pre-training stage 210, the pre-trained BioBERT model 300 is fine-tuned in a fine-tuning stage 220 using the input data 102 and pseudo-labels 122, 124. As will become apparent, the pre-trained BioBERT model 300 is fine-tuned may be fine-tuned to perform various tasks. For instance, the LoT sentence detector 130 uses the BioBert model 300 to generate a corresponding LoT indicator 132 for a respective sequence of terms 104. The LoT indicator 132 is a binary classification that indicates “true” (e.g., ‘1’) when the BioBert model 300 of the LoT sentence detector 130 determines a sequence of terms 104 includes LoT information and indicates “false” (e.g., ‘0” when the LoT sentence detector 130 determines a sequence of terms 104 does not include LoT information. Thereafter, a loss module 240 determines a corresponding LoT indication loss 242 based on comparing the corresponding LoT indicator 132 generated by the BioBert model 300 and the corresponding LoT pseudo-label 122 generated by the regex module 120 (FIG. 1). Here, corresponding LoT indicator 132 and the corresponding LoT pseudo-label 122 are each generated from a same respective sequence of terms 104 whereby the corresponding LoT pseudo-label 122 serves as the ground-truth label. The fine-tuning stage 220 fine-tunes the BioBert model 300 (e.g., updates parameters of the BioBert model 300) based on the LoT indication losses 242 determined for the one or more sequences of terms 104. Thus, the fine-tuning stage 220 fine-tunes the BioBert model 300 to detect whether sequences of terms 104 include LoT information or not.

Referring back to FIG. 1, the processing system 110 trains the multi-label LoT label generator 140 to classify sequences of terms 104 including LoT information. Similarly, the multi-label LoT label generator 140 may include the pre-trained BioBert model such that the processing system 110 trains the BioBert model 300 to classify LoT information into particular classifications. In some examples, the multi-label LoT label generator 140 shares the same BioBert model 300 as the LoT sentence detector 130. In other examples, the multi-label LoT label generator 140 includes a different BioBert model 300 than the LoT sentence detector 130. Notably, the multi-label LoT label generator 140 is a neural network model that classifies the LoT information non-deterministically (in contrast to the regex module 120). The corresponding classification pseudo-labels 124 generated for each respective sequence of terms 104 serve as corresponding ground-truth labels for training the multi-label LoT label generator 140. The LoT classifications 142 output from the multi-label LoT label generator 140 may include a single classification or multiple classifications. For instance, the classification may indicate that LoT information is associated with a second LoT step form the series of LoT steps. On the other hand, the classification may indicate that LoT information is associated a second LoT step or greater (e.g., second LoT step, third LoT step, fourth LoT step, etc.) from the series of LoT steps.

Referring again to FIGS. 2A and 2B, in some implementations, the fine-tuning stage 220 of the training process 200 fine-tunes the BioBert model 300 to predict accurate LoT classifications 142 for sequences of terms 104 including LoT information. For instance, the multi-label LoT label generator 140 uses the BioBert model 300 to generate a corresponding LoT classification 142 for a respective sequence of terms 104. The LoT classification 142 predicts a classification of the respective sequence of terms 104 by indicating a particular LoT step from a series of LoT steps. Thereafter, the loss module 240 determines a corresponding LoT classification loss 244 based on comparing the corresponding LoT classification 142 generated by the BioBert model 300 and the corresponding classification pseudo-label 124 generated by the regex module 120 (FIG. 1). Here, corresponding LoT classification 142 and the corresponding classification pseudo-label 124 are each generated from a same respective sequence of terms 104 whereby the corresponding classification pseudo-label 124 serves as the ground-truth label. The fine-tuning stage 220 fine-tunes the BioBert model 300 (e.g., updates parameters of the BioBert model 300) based on the LoT classification losses 244 determined for the one or more sequences of terms 104. Thus, the fine-tuning stage 220 fine-tunes the BioBert model 300 to classify the particular LoT step associated with LoT information included in a particular sequence of terms 104.

In machine learning, a supervised machine learning model requires large amounts of labeled training data. However, labeling large amounts of data is time consuming and expensive as it requires manual annotation by subject matter experts. In contrast, the fine-tuning stage 220 of the training process 200 utilizes weakly annotated data to train (e.g., semi-supervised training data) to train the BioBert model 300. That is, the fine-tuning stage 220 relies on the pseudo-labels 122, 124 generated by regular expression rules of the regex module 120 (FIG. 1).

Referring back to FIG. 1, the weak supervision labeling model 150 is configured to generate an aggregation of training data 152 to train the multi-label LoT classifier. In particular, the weak supervision labeling model 150 obtains a large amount of training data from various sources such as the LoT classifications 142 generated by the multi-label LoT label generator 140, the pseudo-labels 122, 124 generated by the regex module 120, and externally labeled training data included in the input data 102. Here, the externally labeled training data included in the input data 102 may include labeled training data from external sources such as commercial vendor websites. Thus, the externally labeled training data may represent a small portion of data that is already labeled by a human annotator.

To that end, the weak supervision labeling model 150 may aggregate the labels from these sources using a weak supervision technique that selects a corresponding pseudo-label for each respective sequence of terms 104 based on a maximum vote received by the label (e.g., MultilabelVoter). That is, for each respective sequence of terms 104, the weak supervision labeling model 150 may receive one or more different labels from human annotators, the regex module 120, and/or the LoT sentence detector 130. Therefore, the weak supervision labeling model 150 may employ an external MultilabelVoter model that determines a confidence value associated with the label from each source and selects the label having the highest confidence value. Subsequently, the weak supervision labeling model 150 outputs the aggregated training data 152 to the multi-label LoT classifier 160. Similarly to the multi-label LoT label generator 140, the multi-label LoT classifier 160 includes the pre-trained BioBert model 300. The pre-trained BioBert model 300 may be the same model as the LoT sentence detector 130 and/or multi-label LoT label generator 140 or a different model. Thereafter, the loss module 240 determines a corresponding LoT classification loss 244 based on comparing the corresponding LoT classification 142 generated by the multi-label LoT classifier 160 using the BioBert model 300 and the corresponding pseudo-label from the aggregated training data 152. That is, the fine-tuning stage 220 trains the multi-label LoT classifier 160 of the BioBert model 300 using pseudo-labels from the aggregated training data 152. The aggregated training data 152 may include pseudo-labels from human annotated data included in the input data 102, LoT classifications 142, and/or classification pseudo-labels 124. In contrast, the fine-tuning stage 220 trains the multi-label LoT label generator 140 of the BioBert model 300 with ground-truth labels including only the classification pseudo-labels 124. Simply put, the multi-label LoT label generator 140 is trained to classify LoT information based on classification pseudo-labels 124 while the multi-label LoT classifier 160 is trained to classify LoT information based on the aggregation of training data 152.

Referring again to FIGS. 2A and 2B, the multi-label LoT classifier 160 similarly may use the BioBert model 300 to predict corresponding LoT classifications 142 for respective sequences of terms 104. Thereafter, the aggregated labels (e.g., aggregated data). The multi-label LoT classifier 160 can be a fine-tuned BioBert model that is trained using the labels generated by the weak supervision labeling model 150. The aggregated data can be used as new training data (e.g., input data 102) for use in training, for example, the multi-label LoT classifier 160. Thereafter, the loss module 240 determines a corresponding LoT classification loss 244 based on comparing the corresponding LoT classification 142 generated by the multi-label LoT classifier 160 using the BioBert model 300 and the corresponding pseudo-label from the aggregated training data 152. That is, the fine-tuning stage 220 trains the multi-label LoT classifier 160 of the BioBert model 300 using pseudo-labels from the aggregated training data 152. The aggregated training data 152 may include pseudo-labels from human annotated data included in the input data 102, LoT classifications 142, and/or classification pseudo-labels 124. In contrast, the fine-tuning stage 220 trains the multi-label LoT label generator 140 using the BioBert model 300 with ground-truth labels only from the classification pseudo-labels 124.

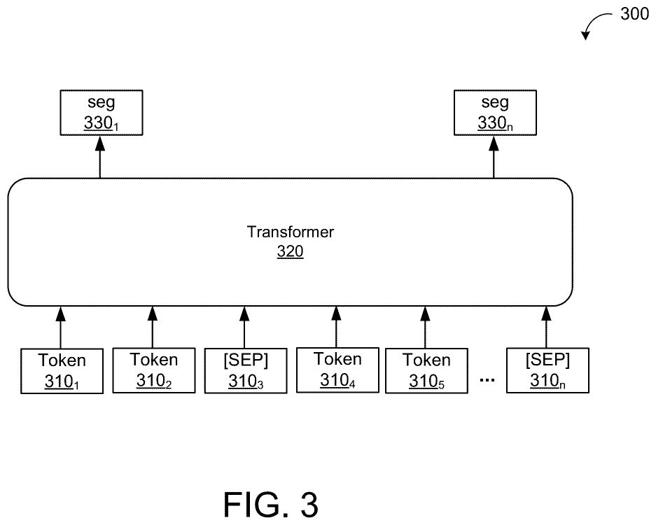

Referring now to FIG. 3, in some implementations, the BioBert model 300 includes a transformer 320 that process the sequences of terms 104 to identify and extract features related to LoT information from clinical trial documents. Text segments derived from clinical trial documents (e.g., the sequences of terms 104) are tokenized into a series of tokens 310 (some of which may include special tokens SEP which indicate a separation between adjacent sentences) for input into the transformer 320. These text segments can be generated using a Coherence-Aware Text Segmentation (CATS) framework. The transformer 320 can take various forms including, for example, a transformer-based machine learning model or ensemble of models for natural language processing (NLP) such as BERT. Thus, the transformer 320 may include a stack of multi-headed self-attention layers. For instance, the stack of multi-headed self-attention layers may include transformer layers or conformer layers in lieu of transformer layers. NLP can be applied to various biomedical applications such as for use in the identification of tumor status from unstructured medical resonance imaging (MM) reports, lung cancer stages from pathology reports, cancer stage information from narrative electric health record (EHR) data to extract features such as disease, age, sex, and/or race attributes of patients. The output of the transformer 320 can include a series of segments 330i-n that can be further labeled using the various techniques described in FIG. 1.

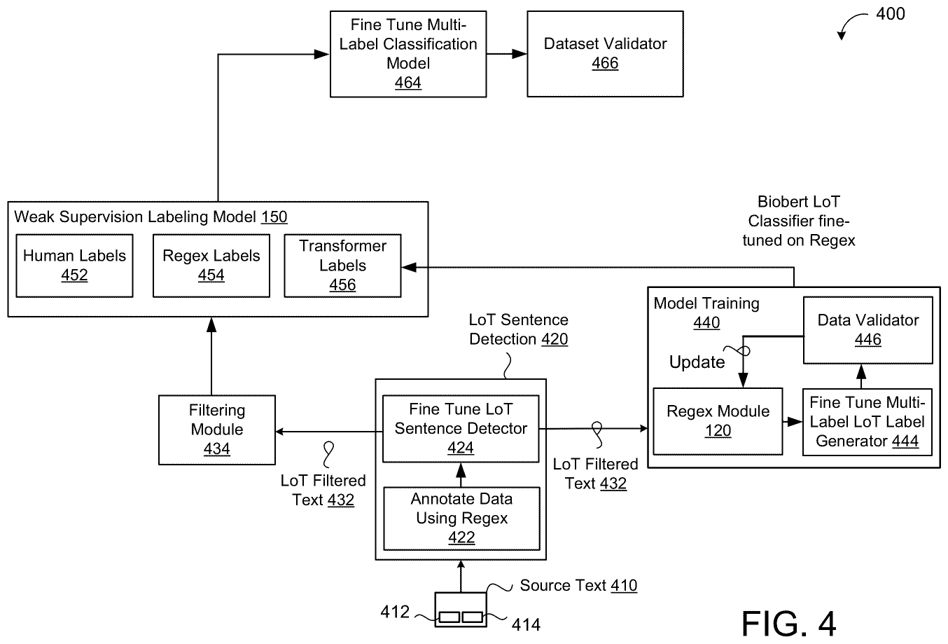

FIG. 4 is a process flow diagram 400 for training the one or more machine learning components 170 (FIG. 1). The training process begins with a series of source texts 410. The source texts 410 may include information such as study inclusion criteria 412 and/or a study official title 414. Thereafter, LoT detection steps 420 process the source texts 410. For example, at step 422, the regex module 120 annotates the source texts 410 using initial regex expression rules. Thereafter, at step 424, the pre-trained BioBERT model can be fine-tuned based on the LoT indicators 132 and the corresponding LoT pseudo-label 122 to detect line of therapy sentences. Optionally, at step 432, sentences from the source text 410 that include LoT information may be filtered or extracted from the source text 410. For instance, only studies from the clinical documents that have at least LoT sentence may be filtered and used to train the machine learning components 170.

The extracted sentences having LoT information can be used to further model training 440 (e.g., training of the multi-label LoT label generator 140). More specifically, at step 442, the regex module 120 may annotate the extracted sentences using initial regular expression rules. Thus, a BioBERT model 300 of the multi-label LoT label generator 140 may be further fine-tuned, at step 444, based on determining LoT classification losses 244. A validation of the classified LoT may include, at step 446, determining whether the classification is proper and update the initial regular expression rules of the regex module 120 accordingly.

In some examples, the extracted study is annotated using the weak supervision labeling model 112d that was previously described in FIG. 1. As previously described, the weak supervision labeling model 150 may generate the aggregation of training data 152 including external source LoT classifications labeled at the study (e.g., human) level 452, pseudo-labels 122, 124 generated by the regex module 120 (e.g. regex labels 545), and labels generated by the multi-label LoT label generator 140). The line of therapy classification model 456 can also be further trained through the labels generated by the BioBert model 300. The weak supervision labeling model 150 generates the aggregation of training data 152 using weak supervision techniques as described in FIG. 1. The BioBERT model 300 of the multi-label LoT classifier 160 may be further fine-tuned through training, at step 464, using the aggregation of training data 152 to classify LoT information.

In some implementations, the classified LoT information may be validated, at step 466, using various datasets. That is, the validated LoT information is validated manually against various annotated studies. In some examples, precision and recall metrics can be utilized to evaluate the identified lines of therapy. A precision score quantifies the number of positive class predictions that actually belong to the positive class. A recall score quantifies the number of positive class predictions made out of all positive examples in the dataset. An F1 score provides a single score that combines both the precision score and the recall score. By way of example, use of the machine learning component(s) 170 as described herein can result in precision and recall scores, including but not limited to, a precision score of about 0.81, a recall score of about 0.97, and an F1 score of about 0.88.

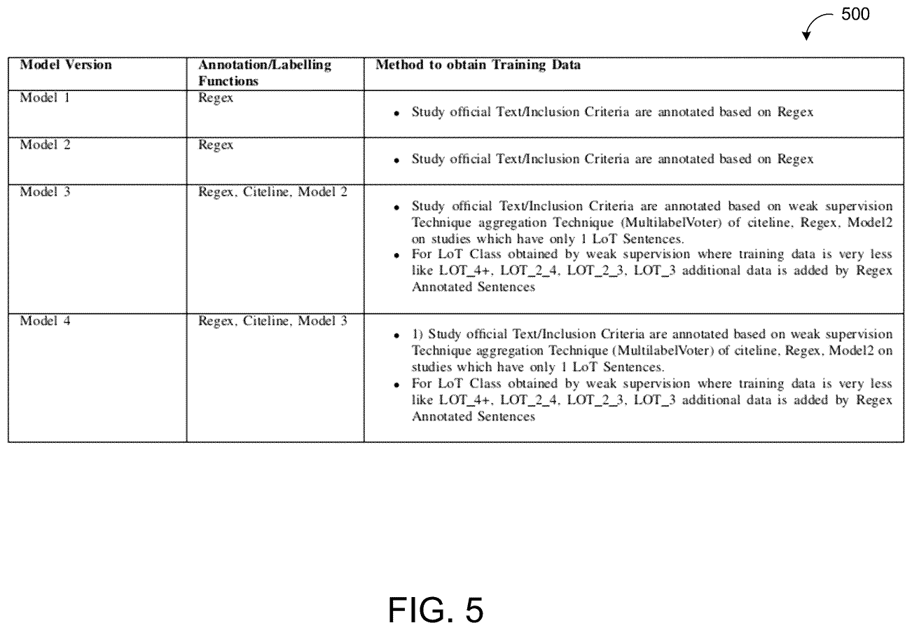

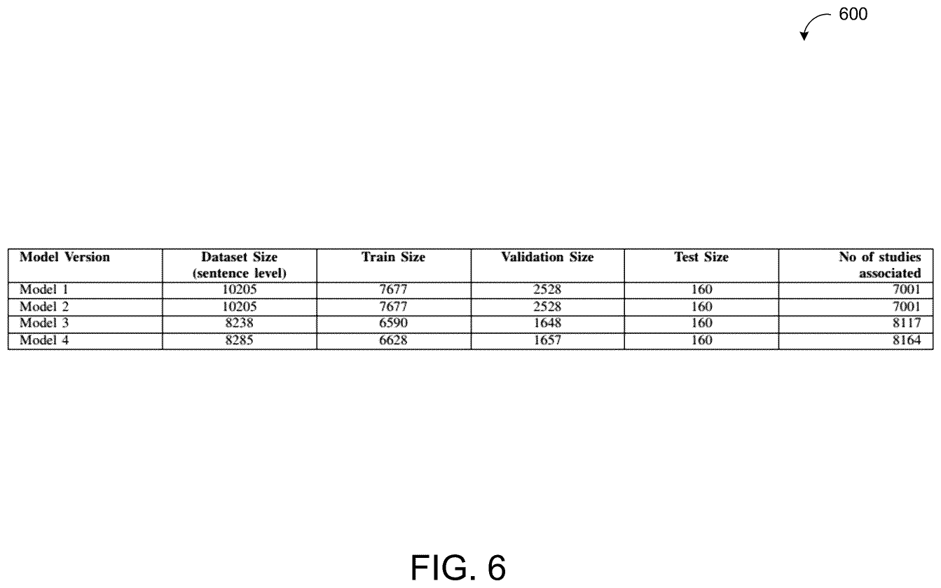

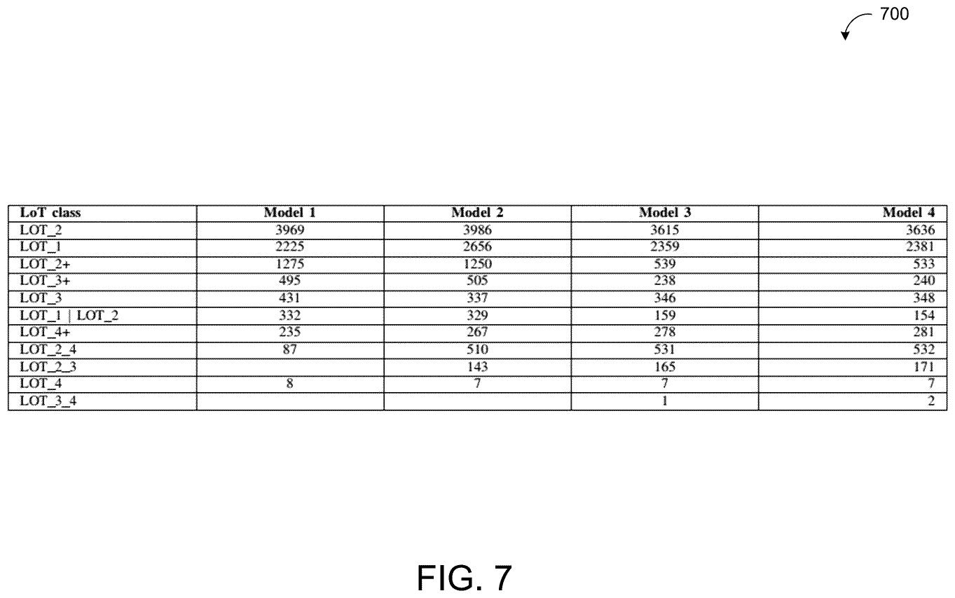

To validate the functionality of the system 100, different versions of the system 100 were generated and validated using different training datasets and annotation/labeling functions. By way of a first example, clinical trial data was collected from a publicly available real-time research and development data for the pharmaceutical industry (e.g., Citeline). From approximately 8117 studies, approximately 10,205 sentences were extracted. These sentences included official text and inclusion criteria of patients. The sentences were annotated using an initial version of regex module 112a. FIG. 5 is a table 500 providing descriptions of the different versions of system 100 that were generated. “Model 1” was trained using approximately 7,677 annotated sentences, and the validation set size was approximately 2,528. “Model 1” achieved a precision score of approximately 0.62, a recall score of approximately 0.79, and an F1 score of approximately 0.69. FIG. 6 is a table 600 providing the number of data samples for training and test set for the models described in table 500 of FIG. 5. FIG. 7 is a table 700 defining describing the distribution of LoT classifications for different fine-tuned BioBERT models 300 of FIG. 5.

By analyzing the output of the validation set, the regex module 120 was updated and a second version of the model, namely “Model 2.” Model 2 was trained using approximately 7,677 annotated sentences, and the validation set size was approximately 2,528. “Model 2” achieved a precision score of approximately 0.79, a recall score of approximately 0.86, and an F1 score of 0.82. The regex module 120 was updated again based on the output of the validation set for Model 2.

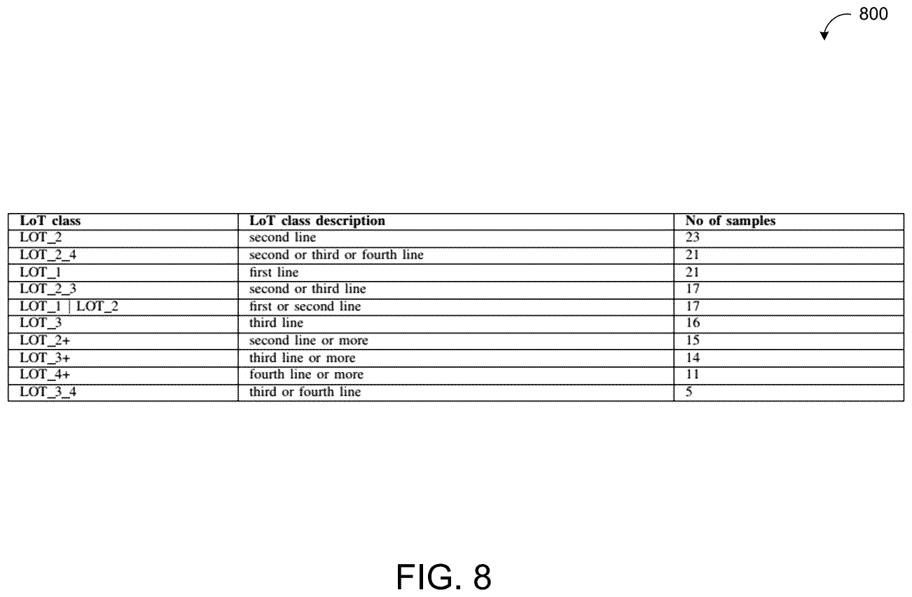

A weak supervision technique using the weak supervision labeling model 150 was used to collect training data for “Model 3.” More specifically, for “Model 3” a set of training data was generated using output from “Model 2.” Another set of training data was generated using the updated regex module 120 as the labeling function. The last set of labeled data was from labels created by an external data source, Citeline. The output from “Model 3” was aggregated using the multi-label LoT classifier 160. “Model 3” achieved a precision score of approximately 0.81, a recall score of approximately 0.95, and an F1 score of approximately 0.87. The pre-trained BioBERT model 300 to develop “Model 4.” the performance of “Model 4” was evaluated using a separated test set of approximately 160 samples. Experts in the clinical trial domain manually annotated these samples. “Model 4” achieved a precision score of approximately 0.81, a recall score of approximately 0.97, and an F1 score of approximately 0.88. FIG. 8 is a table 800 showing the number of data samples in each of the line of therapy classes in the ground truth data.

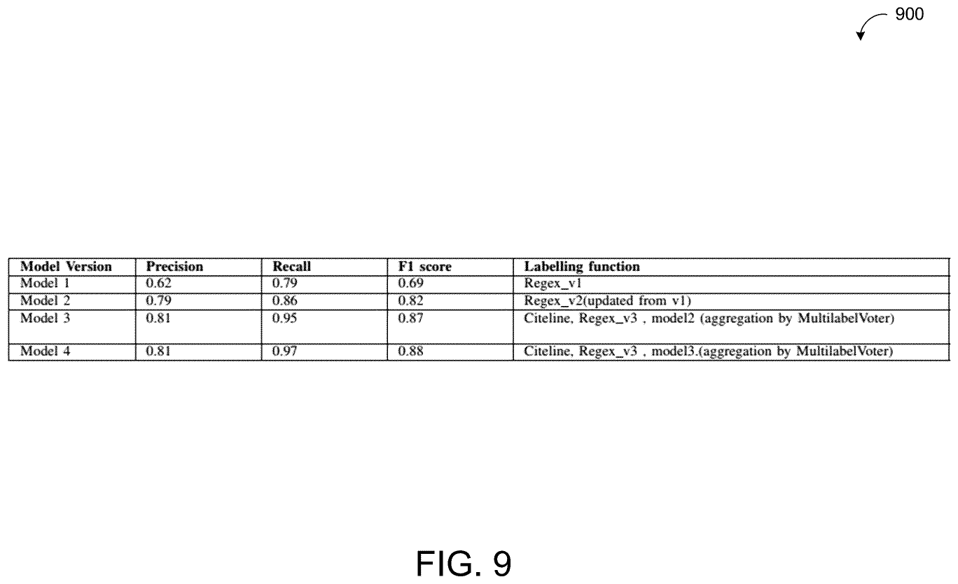

Using a weak supervision-based fine-tuned BioBERT model appears to perform better for the classification of the LoT information than a rule and regular expressions-based model. FIG. 9 is a table 800 showing the precision, recall, and F1 scores of the different models in table 500 of FIG. 5. Based on the performance metrics in table 800, it is clear that the weak supervision-based models improve the recall score. On a validation dataset of approximately 160 studies, the system 100 achieves a precision score of approximately 0.81, a recall score of approximately 0.97, and an F1 score of approximately 0.88.

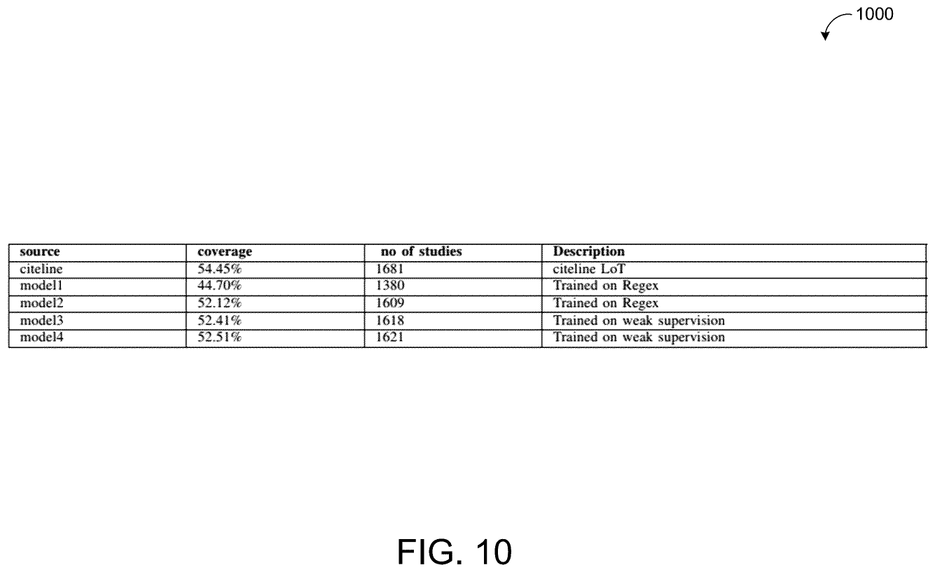

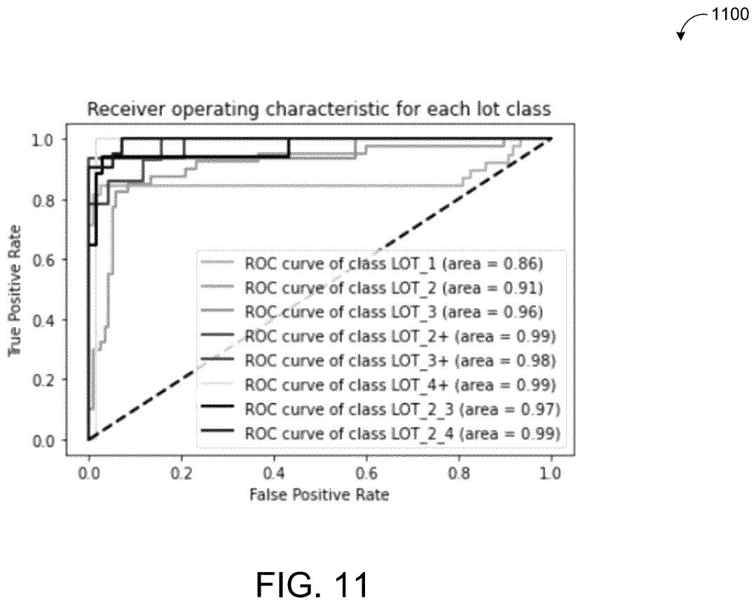

FIG. 10 is a table 1000 that shows a coverage analysis for the various models of table 500 on an untouched dataset (i.e., a dataset unseen by the various models during training). FIG. 11 is a graph 1100 illustrating receiver operating characteristic curves showing the true positive rate versus the false positive rate for each of the line of therapy classes.

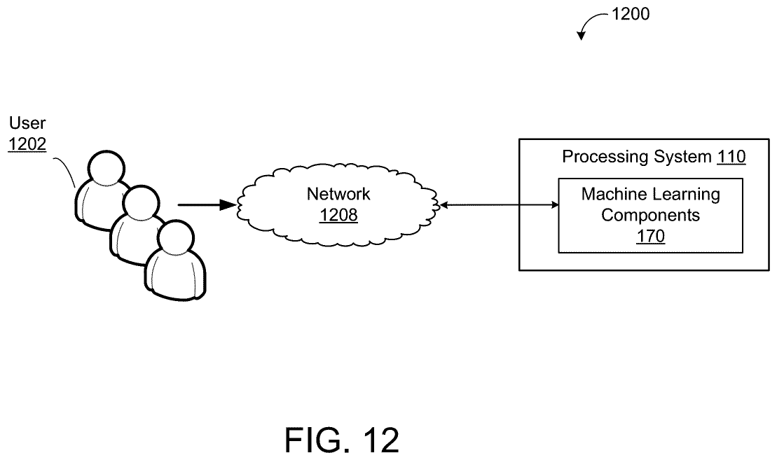

FIG. 12 is a process flow diagram 1200 illustrating a method for classifying LoT information clinical trial documents. In some examples, the trained one or more machine learning components 170 reside and execute on the processing system 110 such that the processing system 110 receives the input data 102 from other computing devices associated with users 1202 in communication with the machine learning components 170. In other examples, the processing system 110 sends the trained one or more machine learning components 170 to the other computing devices associated with the users 1202.

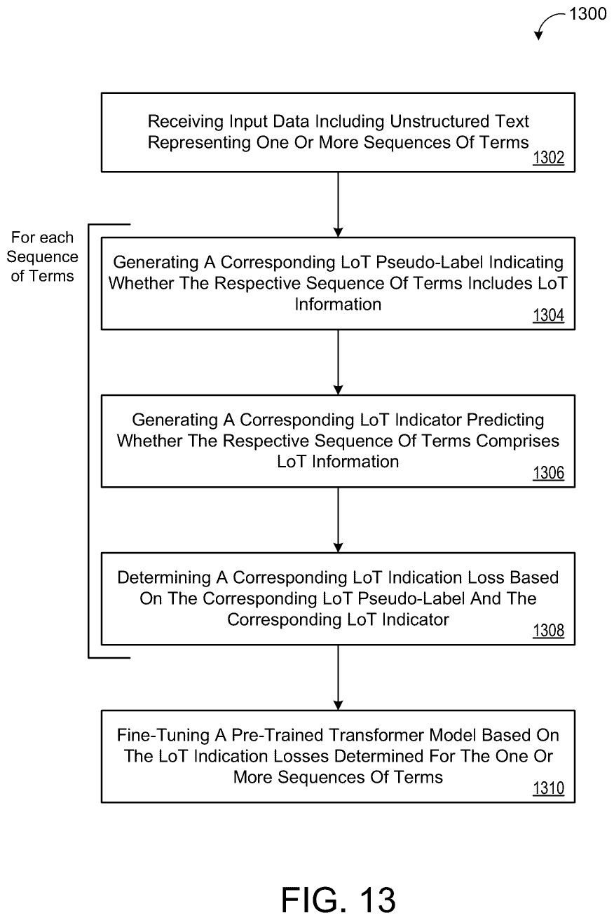

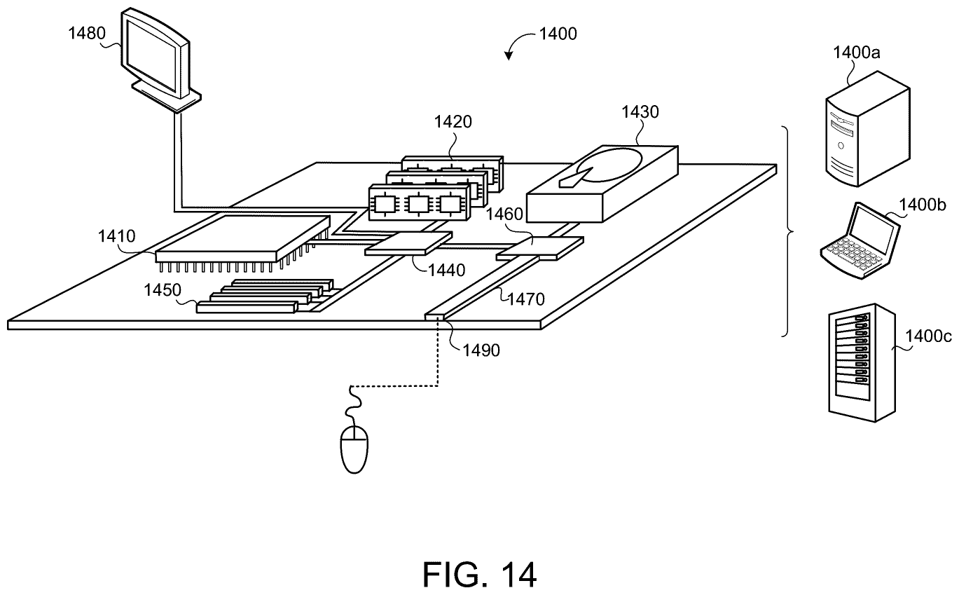

FIG. 13 is a flowchart of an example arrangement of operations for a computer-implemented method 1300 of a weak supervision based deep learning model for LoT identification from clinical documents. The method 1300 may execute on data processing hardware 1410 (FIG. 14) using instructions stored on memory hardware 1420 (FIG. 14). The data processing hardware 1410 and the memory hardware 1420 may reside on the processing system 110 (e.g., user device and/or cloud computing environment) corresponding to the computing device 1400 (FIG. 14).

At operation 1302, the method 1300 includes receiving input data 102 that includes unstructured text representing one or more sequences of terms 104. For each respective sequence of terms 104, the method 1300 performs operations 1304-1308. At operation 1304, the method 1300 includes generating, using regular expression rules, a corresponding line of therapy (LoT) pseudo-label 122 indicating whether the respective sequence of terms 104 includes LoT information. At operation 1306, the method 1300 includes generating, using a pre-trained transformer model 300, a corresponding LoT indicator 132 predicting whether the respective sequence of terms 104 includes LoT information. At operation 1308, the method 1300 includes determining a corresponding LoT indication loss 242 based on the corresponding LoT pseudo-label 122 and the corresponding LoT indicator 132. At operation 1310, the method 1300 includes fine-tuning the pre-trained transformer model 300 based on the LoT indication losses 242 determined for the one or more sequences of terms 104.

FIG. 14 is a schematic view of an example computing device 1400 that may be used to implement the systems and methods described in this document. The computing device 1400 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 1400 includes a processor 1410, memory 1420, a storage device 1430, a high-speed interface/controller 1440 connecting to the memory 1420 and high-speed expansion ports 1450, and a low speed interface/controller 1460 connecting to a low speed bus 1470 and a storage device 1430. Each of the components 1410, 1420, 1430, 1440, 1450, and 1460, are interconnected using various busses, and may be mounted on a common motherboard or in other manners as appropriate. The processor 1410 can process instructions for execution within the computing device 1400, including instructions stored in the memory 1420 or on the storage device 1430 to display graphical information for a graphical user interface (GUI) on an external input/output device, such as display 1480 coupled to high speed interface 1440. In other implementations, multiple processors and/or multiple buses may be used, as appropriate, along with multiple memories and types of memory. Also, multiple computing devices 1400 may be connected, with each device providing portions of the necessary operations (e.g., as a server bank, a group of blade servers, or a multi-processor system).

The memory 1420 stores information non-transitorily within the computing device 1400. The memory 1420 may be a computer-readable medium, a volatile memory unit(s), or non-volatile memory unit(s). The non-transitory memory 1420 may be physical devices used to store programs (e.g., sequences of instructions) or data (e.g., program state information) on a temporary or permanent basis for use by the computing device 1400. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 1430 is capable of providing mass storage for the computing device 1400. In some implementations, the storage device 1430 is a computer-readable medium. In various different implementations, the storage device 1430 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 1420, the storage device 1430, or memory on processor 1410.

The high speed controller 1440 manages bandwidth-intensive operations for the computing device 1400, while the low speed controller 1460 manages lower bandwidth-intensive operations. Such allocation of duties is exemplary only. In some implementations, the high-speed controller 1440 is coupled to the memory 1420, the display 1480 (e.g., through a graphics processor or accelerator), and to the high-speed expansion ports 1450, which may accept various expansion cards (not shown). In some implementations, the low-speed controller 1460 is coupled to the storage device 1430 and a low-speed expansion port 1490. The low-speed expansion port 1490, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 1400 may be implemented in a number of different forms, as shown in the figure. For example, it may be implemented as a standard server 1400a or multiple times in a group of such servers 1400a, as a laptop computer 1400b, or as part of a rack server system 1400c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method that when executed on data processing hardware causes the data processing hardware to perform operations comprising:
receiving input data comprising unstructured text representing one or more sequences of terms;
for each respective sequence of terms:
generating, using regular expression rules, a corresponding line of therapy (LoT) pseudo-label indicating whether the respective sequence of terms comprises LoT information;
generating, using a pre-trained transformer model, a corresponding LoT indicator predicting whether the respective sequence of terms comprises LoT information; and
determining a corresponding LoT indication loss based on the corresponding LoT pseudo-label and the corresponding LoT indicator; and
fine-tuning the pre-trained transformer model based on the LoT indication losses determined for the one or more sequences of terms.

2. The computer-implemented method of claim 1, wherein the operations further comprise:
for each respective sequence of terms:
generating, using the regular expression rules, a corresponding classification pseudo-label indicating a classification of the respective sequence of terms;
generating, using the pre-trained transformer model, a corresponding LoT classification predicting the classification of the respective sequence of terms; and
determining a corresponding LoT classification loss based on the corresponding classification pseudo-label and the corresponding LoT classification; and
fine-tuning the pre-trained transformer model based on the LoT classification losses determined for the one or more sequences of terms.

3. The computer-implemented method of claim 2, wherein the classification of the respective sequence of terms indicates a particular LoT step from a series of LoT steps associated with the respective sequence of terms.

4. The computer-implemented method of claim 1, wherein the input data comprises a plurality of clinical trial documents comprising the unstructured text.

5. The computer-implemented method of claim 1, wherein the operations further comprise updating the regular expression rules based on the corresponding LoT indication loss.

6. The computer-implemented method of claim 1, wherein the pre-trained transformer model comprises a Bidirectional Encoder from Transformers for Biomedical Text Mining (BioBERT) model pre-trained on a corpus of biomedical text data.

7. The computer-implemented method of claim 6, wherein the pre-trained BioBERT model comprises a stack of multi-headed self-attention layers.

8. The computer-implemented method of claim 1, wherein the operations further comprise fine-tuning the pre-trained transformer model using an aggregation of training data including human annotated labels, labels generated using the regular expression rules, and labels generated by the pre-trained transformer model.

9. The computer-implemented method of claim 1, wherein the operations further comprise storing the fine-tuned transformer model in memory hardware in communication with the data processing hardware.

10. The computer-implemented method of claim 1, wherein the operations further comprising transmitting, via a network, the fine-tuned transformer model to one or more computing devices.

11. A system comprising:
data processing hardware; and
memory hardware in communication with the data processing hardware, the memory hardware storing instructions that when executed on the data processing hardware cause the data processing hardware to perform operations comprising:
receiving input data comprising unstructured text representing one or more sequences of terms;
for each respective sequence of terms:
generating, using regular expression rules, a corresponding line of therapy (LoT) pseudo-label indicating whether the respective sequence of terms comprises LoT information;
generating, using a pre-trained transformer model, a corresponding LoT indicator predicting whether the respective sequence of terms comprises LoT information; and
determining a corresponding LoT indication loss based on the corresponding LoT pseudo-label and the corresponding LoT indicator; and
fine-tuning the pre-trained transformer model based on the LoT indication losses determined for the one or more sequences of terms.

12. The system of claim 11, wherein the operations further comprise:
for each respective sequence of terms:
generating, using the regular expression rules, a corresponding classification pseudo-label indicating a classification of the respective sequence of terms;
generating, using the pre-trained transformer model, a corresponding LoT classification predicting the classification of the respective sequence of terms; and
determining a corresponding LoT classification loss based on the corresponding classification pseudo-label and the corresponding LoT classification; and
fine-tuning the pre-trained transformer model based on the LoT classification losses determined for the one or more sequences of terms.

13. The system of claim 12, wherein the classification of the respective sequence of terms indicates a particular LoT step from a series of LoT steps associated with the respective sequence of terms.

14. The system of claim 11, wherein the input data comprises a plurality of clinical trial documents comprising the unstructured text.

15. The system of claim 11, wherein the operations further comprise wherein the operations further comprise updating the regular expression rules based on the corresponding LoT indication loss.

16. The system of claim 11, wherein the pre-trained transformer model comprises a Bidirectional Encoder from Transformers for Biomedical Text Mining (BioBERT) model pre-trained on a corpus of biomedical text data.

17. The system of claim 16, wherein the pre-trained BioBERT model comprises a stack of multi-headed self-attention layers.

18. The system of claim 11, wherein the operations further comprise fine-tuning the pre-trained transformer model using an aggregation of training data including human annotated labels, labels generated using the regular expression rules, and labels generated by the pre-trained transformer model.

19. The system of claim 11, wherein the operations further comprise storing the fine-tuned transformer model in memory hardware in communication with the data processing hardware.

20. The system of claim 11, wherein the operations further comprising transmitting, via a network, the fine-tuned transformer model to one or more computing devices.
